(19) United States
(12) Patent Application Publication
(10) Pub. No.: US 2010/0196555 A1
(43) Pub. Date: Aug. 5, 2010

(54) KIT FOR MAKING A NON-FROZEN DESSERT AND A METHOD OF ASSEMBLY

(76) Inventor: Elizabeth Guidobono, Northville, MI (US)

Correspondence Address: HOWARD & HOWARD ATTORNEYS PLLC, 450 West Fourth Street, Royal Oak, MI, 48067, US

(21) Appl. No.: 12/696,738

(22) Filed: Jan. 29, 2010

Related U.S. Application Data
(60) Provisional application No. 61/148,636, filed on Jan. 30, 2009.

(51) Int. Cl. B65D 81/32; G01N 33/02

(52) U.S. Cl. 426/113; 426/232

(57) ABSTRACT

A kit for making a non-frozen dessert capable of being transported and stored without sacrificing freshness comprises a carton, a pan disposed in the carton for forming the non-frozen dessert, and a plurality of ingredients disposed in the carton for making the non-frozen dessert. The plurality of ingredients includes a crust ingredient for disposing on the pan prior to baking the non-frozen dessert. The plurality of ingredients also includes a filling comprising a sugar and spice ingredient, a butter ingredient, and a fresh fruit ingredient. The crust ingredient, the sugar and spice ingredient, and the butter ingredient are each individually packed in hermetically sealed packages and the fresh fruit ingredient is individually packed in a vacuum sealed package and includes at least one preservative for ensuring freshness. The kit for making a non-frozen dessert also comprises a thermometer capable of measuring a temperature of the filling.

This application claims priority to U.S. Provisional Patent Application Ser. No. 61/148,636 filed Jan. 30, 2009.

### FIELD OF THE INVENTION

The subject invention relates to a kit for making a non-frozen dessert capable of being transported and stored without sacrificing freshness and a method of assembling the kit.

### BACKGROUND OF THE INVENTION

As the pace of life has increased, so has the need to minimize the amount of time and effort spent on daily tasks, such as food preparation. The market has responded to this need with food products, such as pre-cooked, pre-prepared, and other food products that taste good and can be prepared in a in a minimal amount of time with a minimal amount of effort. In fact, pre-cooked, pre-prepared, and other such food products are common items on grocery store shelves and in grocery store refrigerators and freezers. Typically such food products are eaten as packaged or after a brief heating period. One such food product is a dessert product.

The availability of the dessert product has increased and the popularity of making homemade desserts has decreased. Making homemade desserts from scratch with fresh ingredients, i.e., baking, has become a lost art. Family recipes that were developed over decades and tweaked to perfection have been lost. The nuances of baking homemade desserts in a particular oven to achieve homemade taste are no longer learned through trial and error. Although the dessert product can be prepared with little or no time and effort, the dessert product falls short of homemade desserts in many ways.

One dessert product, a pre-cooked dessert product takes little or no time and effort to prepare. By pre-cooked, it is meant that the dessert product is fully cooked and sold in a ready-to-consume state with little or no additional cooking, other than reheating. The pre-cooked dessert product comes in two forms (1) frozen and (2) non-frozen. If the pre-cooked dessert product is of the frozen form, additives required for, and the freezing process itself, impact consistency and product quality. If the pre-cooked dessert product is of the non-frozen form, perishability is a concern. As such, the pre-cooked dessert product has many shortcomings, it is not fresh, does not taste homemade, and is often perishable.

The market responded to the shortcomings of the pre-cooked dessert product with a pre-prepared dessert product. By pre-prepared, it is meant that the dessert product is prepared, i.e., put together, but not fully cooked and sold. Accordingly, the pre-prepared dessert product must be cooked by the consumer prior to consumption. The pre-prepared dessert product is typically sold frozen. Likewise, additives required for, and the freezing process itself, impact consistency and the product quality of the pre-prepared dessert. Take a pre-prepared meal purchased by a consumer as an example, the pre-prepared meal being chicken and dumplings, the consumer makes dough, adds cans of filler/soup, and viola dinner. However, the meal in this example still tastes chemical, processed, and not “fresh”, not homemade. Must be able to sit on a shelf for 6 months without changed (this is a big point about shelf life, when comparing freshness). Although the pre-prepared dessert product can be considered an improvement over the pre-cooked dessert product, the pre-prepared dessert product is not fresh and does not taste homemade.

In addition to the shortcomings mentioned above, both the pre-cooked dessert product and the pre-prepared dessert product do not provide a baker with the satisfaction associated with the preparation and baking of homemade desserts, nor do they provide an eater with the freshness and taste of homemade desserts prepared from scratch. Although pre-cooked and pre-prepared dessert products make our hectic life-styles seem a bit easier, there remains the need for a dessert product that can be effectively prepared with a minimal amount of time and effort and provides satisfaction associated with preparing and serving fresh, homemade desserts, as well as fresh, homemade taste.

### SUMMARY OF THE INVENTION

Accordingly, the invention provides a kit for making a non-frozen dessert capable of being transported and stored without sacrificing freshness. The kit comprises a carton, a pan disposed in the carton for forming the non-frozen dessert, and a plurality of ingredients disposed in the carton for making the non-frozen dessert. The plurality of ingredients includes a crust ingredient for disposing on the pan prior to baking the non-frozen dessert and a filling for disposing on the crust ingredient prior to baking the non-frozen dessert. The filling comprises a sugar and spice ingredient, a butter ingredient, and a fresh fruit ingredient. The crust ingredient, the sugar and spice ingredient, and the butter ingredient are each individually packed in hermetically sealed packages and the fresh fruit ingredient is individually packed in a vacuum sealed package and includes at least one preservative for ensuring freshness. The kit for making a non-frozen dessert also comprises a thermometer capable of measuring a temperature of the filling.

The subject invention also provides a method of assembling the kit described above. The method comprises the steps of providing the pan, providing the crust ingredient, providing the filling comprising the sugar and spice ingredient, the butter ingredient, and the fresh fruit ingredient that includes at least one preservative for ensuring freshness, providing the thermometer capable of measuring the temperature of the filling, and assembling the pan, the crust ingredient, the filling, and the thermometer in the carton to form the kit.

Accordingly, the kit can be assembled efficiently and freshness can be maintained. In addition, the kit allows the consumer to make and enjoy non-frozen desserts with fresh, homemade taste in a minimal amount of time and with a minimal amount of effort.

Other systems, devices, features, and advantages of the disclosed kit for making a non-frozen dessert will be or will become apparent to one with skill in the art upon examination of the following figures and detailed description. All such additional systems, devices, features, and advantages are intended to be included in this description and are intended to be protected by the accompanying claims.

### BRIEF DESCRIPTION OF THE DRAWINGS

Further objects and advantages of the present invention can be found in the detailed description of the preferred embodiments when taken in conjunction with the accompanying drawings in which:

FIG. 1 is a perspective view of various components of the kit for making a non-frozen dessert;

FIG. 2 is a perspective view of the kit for making a non-frozen dessert after assembly;

FIG. 3A is a partial perspective view of a thermometer inserted into the non-frozen dessert in an uncooked state;

FIG. 3B is a partial perspective view of a thermometer inserted into the non-frozen dessert and indicating that the non-frozen dessert is in a cooked state;

FIG. 3C is a perspective view of a thermometer;

FIG. 3D is a partial cross-sectional view of the thermometer inserted into the non-frozen dessert illustrating a spring element in a compressed position;

FIG. 3E is a partial cross-sectional view of the thermometer inserted into the non-frozen dessert illustrating the spring element in a released position with an indicator extended therefrom;

FIG. 4 is a flow chart generally illustrating the steps of providing a kit for making a non-frozen dessert;

FIG. 5 is a schematic diagram illustrating a method of manufacturing a kit for making a non-frozen dessert; and

FIG. 6 is a flow chart illustrating the steps for making the non-frozen dessert with the kit for making a non-frozen dessert.

### DESCRIPTION OF THE INVENTION

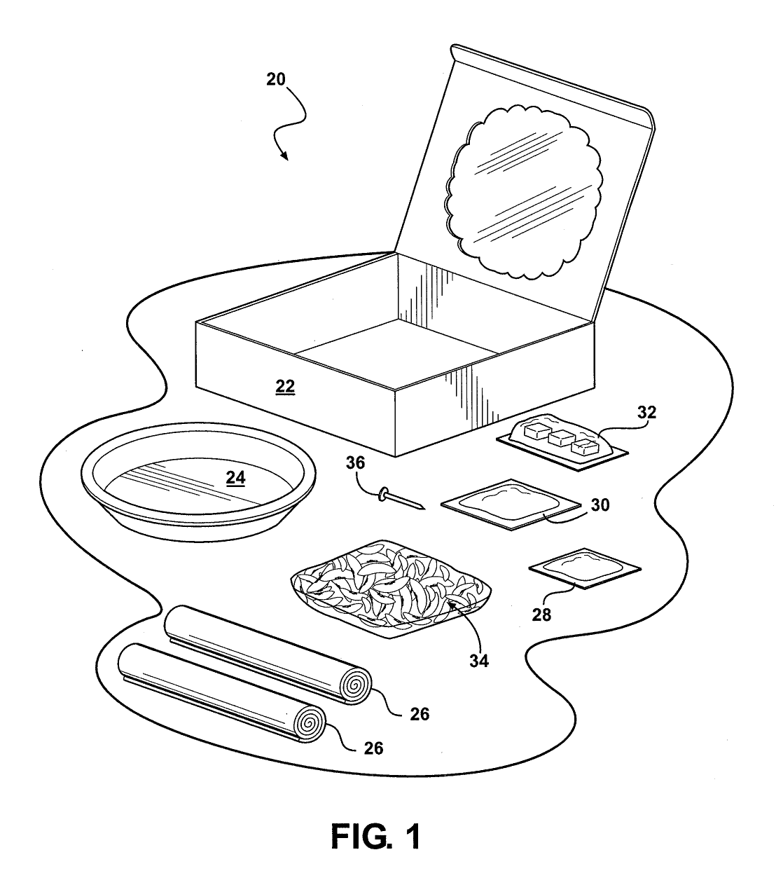

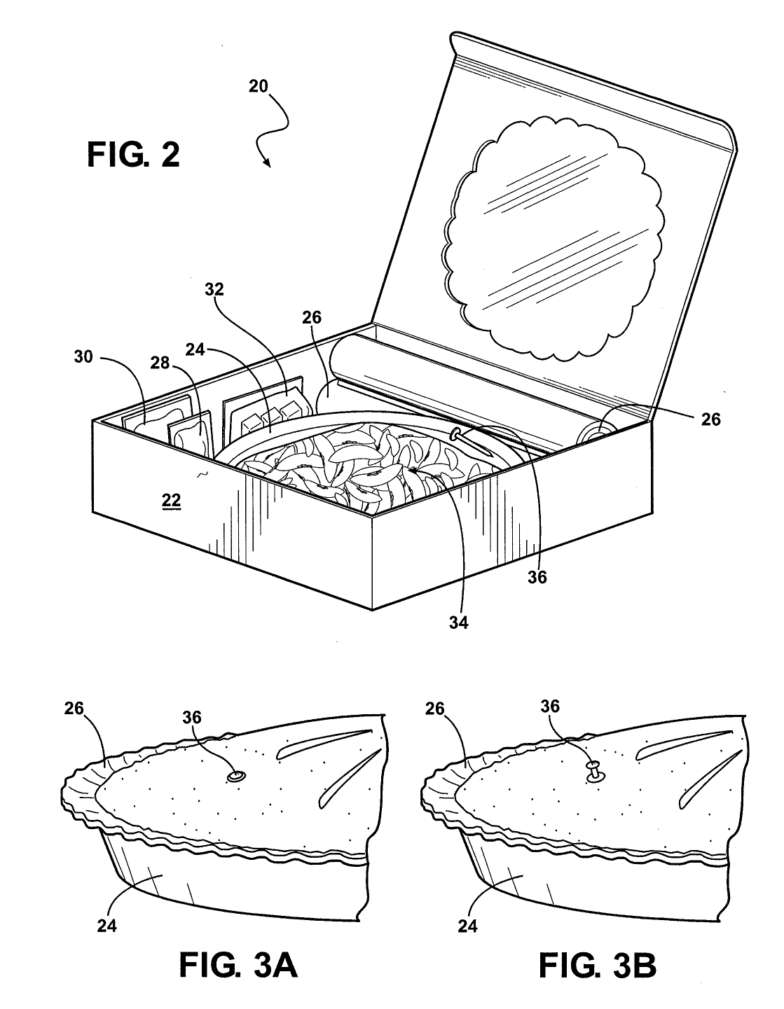

Referring to the Figures, like numerals indicate like parts throughout the several Figures. Referring now to FIGS. 1 and 2, the invention provides a kit 20 for making a non-frozen dessert, herein referred to as the kit 20, capable of being transported and stored without sacrificing freshness. The kit 20 comprises a carton 22, a pan 24 disposed in the carton 22 for forming the non-frozen dessert, and a plurality of ingredients disposed in the carton 22 for making the non-frozen dessert. The non-frozen dessert is herein referred to as the dessert.

The carton 22 can be any type of carton 22 known in the art. The carton 22 is designed to be (1) aesthetically pleasing and (2) to contain a wide variety of components which are assembled for efficient preparation of the dessert by the consumer. The carton 22 is constructed from paperboard, or other materials known in the art, on conventional equipment to form a multi-sided, square or rectangular enclosure. In one embodiment the carton 22 is a multisided, pentagonal enclosure, which has a house-like profile. In a preferred embodiment, the carton 22 includes a window. The window may be left open, formed by a die cut of the carton blank or it may be covered with a transparent film. As such, the construction of carton 22 allows the manufacturer to use conventional and high volume filling equipment and techniques.

The kit 20 includes the pan 24 for forming the dessert, the pan 24 included in the kit 20 is specific to a particular type of dessert. The pan 24 is constructed from aluminum, cast iron, ceramic, copper, earthen ware, glass, non-stick coated steel, Pyrex, silicone, various metals and coated metals, and other materials know in the art. Typically, the pan 24 is of circular configuration having a bottom, a side, and an open top. The bottom has a bottom diameter and the top has a top diameter which is larger than the bottom diameter such that the side slopes outward to a rim which supports a crust. Typically, the top diameter of the pan 24 is about 10 inches and a depth of the pan 24 is about 1.5 inches. Although the pan 24 typically has circular configuration, a top diameter of 9 inches, and a depth of 1.5 inches, other configurations, diameters, and depths have been contemplated. For example, pans having diameters of 6 inches, 8 inches, 9 inches, and even greater diameter have been contemplated. Selection of the pan 24 impacts the look and the taste of the prepared dessert, i.e., proper pan 24 selection provides an aesthetically pleasing, dessert with a tender yet crispy crust, and with fresh fruit slices that hold their shape and melt in ones mouth.

As described above, the kit 20 includes the plurality of ingredients. The plurality of ingredients includes a crust ingredient 26 for disposing on the pan 24 prior to baking the dessert and a filling for disposing on the crust ingredient 26 prior to baking the non-frozen dessert. Other ingredients, such as a sugar ingredient 28 may be optionally included. The filling comprises a sugar and spice ingredient 30, a butter ingredient 32, and a fresh fruit ingredient 34.

The crust ingredient 26 is included for disposing on the pan 24 prior to baking the dessert such as a pie. The crust ingredient 26 comprises pre-mixed dough, a recipe for which is selected specifically for the kit 20. The pre-mixed dough is rolled into a round sheet having a thickness and a diameter sufficient to cover the pan 24. Typically, the premixed dough is rolled to a thickness of from about 0.1 to about 0.75 inches, and more typically from 0.15 to 0.35 inches. Typically, the premixed dough is rolled to a diameter of about 8 inches, more typically about 10 inches, and most typically about 12 inches. After the crust ingredient 26 is rolled into a round sheet, the round sheet is typically dusted with flour and rolled-up to conserve space in the carton 22. However, in one embodiment, the crust ingredient 26 in the form of a round sheet is placed directly on the pan 24.

The kit 20 may include two packages of the crust ingredient 26 for a dessert such as a pie having a both a top crust and a bottom crust. When the kit 20 includes two packages of the crust ingredient 26, the sugar ingredient 28 is typically included to sprinkle over the top crust of the dessert.

Alternatively, the kit 20 may include only one package of the crust ingredient 26 for an open top pie requiring only a bottom crust. In one embodiment, the kit 20 includes one of the crust ingredient 26 and a crunch crust ingredient. Typically, the crunch crust ingredient is a lumpy powder which comprises a mixture of flour, sugar, and other ingredients. The crunch crust ingredient is typically sprinkled on the top of the prepared dessert in lieu of the crunch crust ingredient 26 prior to baking the prepared dessert. After baking, the dessert having a crunchy, crumby top crust results. In addition to, or in lieu of, the crunch crust ingredient 26, the kit 20 may include a topping, such as caramel or cream.

Typically, the crust ingredient 26 is individually packaged in a hermetically sealed package, i.e., a package that is airtight, such that the dough does not dehydrate. Should the kit 20 include the crunch crust ingredient, the crunch crust ingredient is typically individually packaged in a hermetically sealed packet. The crunch crust ingredient is packaged in the hermetically sealed package to maintain freshness.

The plurality of ingredients also includes the fresh fruit ingredient 34. The fresh fruit ingredient 34 comprises at least one of apples, bananas, blackberries, blueberries, cherries, cranberries, grapes, lemons, limes, melons, orange, peaches, pears, pineapple, plums, pomegranate, pumpkin, raspberries, rhubarb, and strawberries. It is also contemplated that the fresh fruit ingredient 34 can comprise various creams, custards, chocolate, and other such fillings. Preferably, the fresh fruit ingredient 34 comprises at least one of apple, banana, blackberry, blueberry, cherry, chocolate, lime, peach, pumpkin, strawberry, raspberry, and rhubarb. Most preferably, the fresh fruit ingredient 34 comprises apples.

In a preferred embodiment, the fresh fruit ingredient 34 comprises apples. Fresh apples should be used to prepare the fresh fruit ingredient 34. The fresh apples should be stored at a temperature of from about 30° to about 40° F., with a relative humidity of about 90 percent and some air circulation. Such storage conditions are necessary because the fresh apples are not dead at the time of harvest. They remain a living, respiring organism and continue to take in oxygen and give off carbon dioxide and another gas, ethylene. Since the fresh apple is no longer receiving nutrients from the tree but is still respiring, it must use up the food it has stored over the growing season. As this food is gradually used up during storage, the sugar, starch, and acid content of the apple changes. Eventually the tissues break down, water is lost, and the apple withers and decays. These storage conditions slow those natural events as much as possible.

Typically the fresh fruit ingredient 34 is cut, cleaned and prepared prior to packaging. In one embodiment where the fresh fruit ingredient 34 comprises apples, the apples are skinned, cored, and sliced to size. In this embodiment, an apple slice must be cut to a certain size such that a thermometer 36 can be firmly inserted into the apple slice without the slice breaking or the thermometer 36 working its way loose during preparation or baking so that a temperature of the filling can be measured accurately. The thermometer 36 will be described in detail below.

In addition, the fresh fruit ingredient 34 is typically treated with a preservative during preparation and prior to being packaged. The preservative is selected to maintain freshness of the fresh fruit ingredient 34 and to prevent browning and improve the perishability of the fresh fruit ingredient 34. Any preservative known in the art can be used to treat the fresh fruit ingredient 34. Typically, the fresh fruit ingredient 34 comprises a carboxylic acid. A suitable type of carboxylic acid is a sugar acid. Preferably, the preservative is ascorbic acid.

Once the fresh fruit ingredient 34 is prepared, the fresh fruit ingredient 34 is individually packaged. The fresh fruit ingredient 34 can be packaged in semi-permeable packages, hermetically sealed packages, or vacuum sealed packages. Semi-permeable packages can produce gas environments with at least 2% oxygen for the natural respiration of fruit tissues. The level of oxygen as well as carbon dioxide is altered by the natural respiration (i.e., consumption of oxygen and generation of carbon dioxide) of the fruit pieces and the permeability of the package to oxygen and carbon dioxide. Hermetically sealed packages are packages that, for practical purposes, are airtight. Vacuum sealed packages are hermetically sealed packages to which a vacuum has been applied to remove substantially all of the air form the package. Typically, the fresh fruit ingredient 34 is sealed in a vacuum sealed package. However, the fresh fruit ingredient 34 can be provided in semi-permeable packages, hermetically sealed packages, or vacuum sealed packages depending on the composition of the fresh fruit ingredient 34. For example, if the fresh fruit ingredient 34 comprises blueberries, a semi-permeable package could be used.

Once the fresh fruit ingredient 34 is packaged, it is preferred the fresh fruit ingredient 34 not be frozen because freezing the fruit negatively impacts the fresh taste of the dessert in its prepared form, i.e., product quality. Preferably, the fresh fruit ingredient 34 is stored and maintained at a temperature of from about 30 to about 75° F. More preferably, the fresh fruit ingredient 34 is stored and maintained at a temperature of from about 30.2 to about 55° F. Most preferably, the fresh fruit ingredient 34 is stored and maintained at a temperature of from about 30.5 to about 45° F. Storage at temperatures of from about 31° F. to about 35° F. provide the greatest delay in the normal ripening and aging process of the fresh fruit ingredient 34.

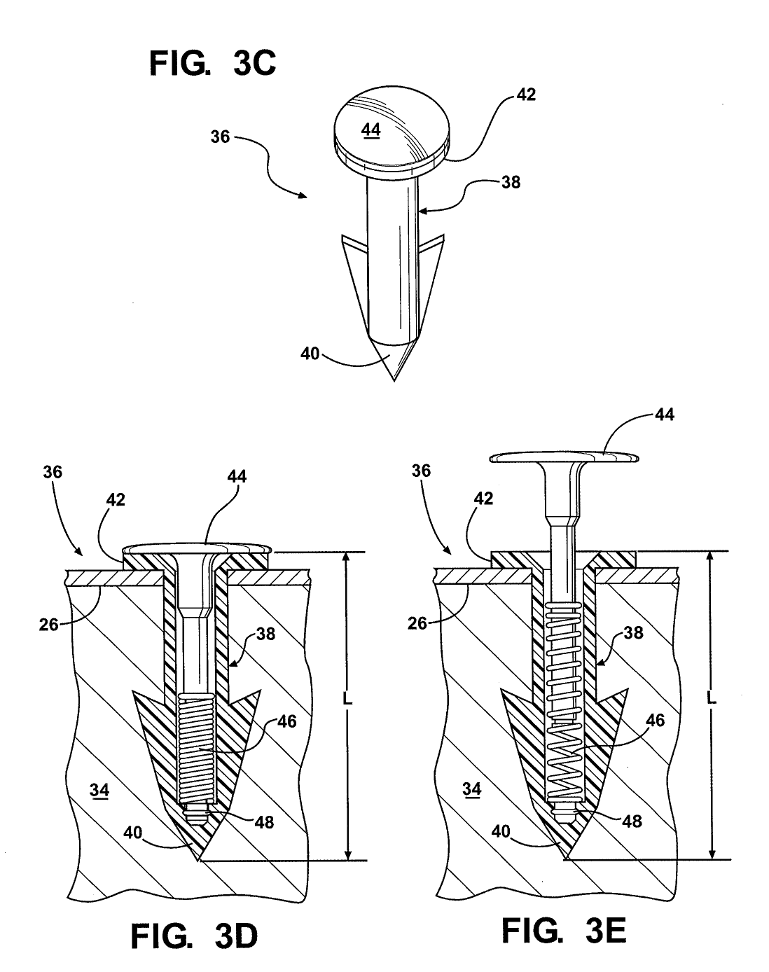

Referring now to FIGS. 3A-3E, the kit 20 also includes the thermometer 36 capable of measuring a temperature of the filling comprising the fresh fruit ingredient 34. The thermometer 36 can be any type of thermometer such as a pop-up thermometer, a dial thermometer, a digital thermometer, or even a wired or a wireless thermocouple. The type of thermometer 36 is selected based on a type of dessert. Thermometers, such as a pop-up thermometer are commercially available from companies such as Volk Enterprises of Alpharetta, Ga. Preferably, the thermometer 36 is a pop-up thermometer.

In one embodiment the kit 20 includes a thermometer 36 comprising a barrel 38 defining a tubular configuration. The barrel has a closed end 40, an open end 42, and a length L. The thermometer 36 also comprises an indicator 44, a release element 48 having a pre-determined melting temperature, and a spring element 46 disposed within the barrel 38. The spring element 46 abuts the closed end 40 of the barrel and is movable between a compressed position, as shown in FIG. 3D, and a released position, as shown in FIG. 3E. The position of the spring element 46 is dependent on a temperature of the release element 48. The release element 48 holds the spring element 46 in the compressed position if the temperature of the release element 48 is less than the pre-determined melting temperature. When the temperature of the release element 48 exceeds the pre-determined melting temperature, the spring element 46 is released thereby extending the indicator 44 out of the open end 42 of the barrel 38 to indicate that the dessert is in a cooked state.

If the thermometer 36 is a pop-up thermometer, the length L of the barrel is selected based on a type of dessert, e.g., based on the fresh fruit ingredient 34 and the crust ingredient 26 of the type of dessert. For example, in an embodiment of the kit 20 for making apple pie having a bottom crust and a top crust, i.e., two of the crust ingredient 26, a pop-up thermometer 36 having a barrel having a length L of about 1.0 inch is preferred. In another embodiment of the kit 20 for making apple pie having a bottom crust, i.e., one of the crust ingredient 26, and the crunch crust ingredient, a pop-up thermometer 36 having a barrel having a length L of about 1.75 inches is preferred. Generally, the thermometer 36 of the present invention has a barrel having a length L of from about 0.5 to about 2.5 inches.

If the thermometer 36 is a pop-up thermometer, the release element 48 having the pre-determined melting temperature is selected based on the type of dessert. Said differently, the release element 48 having the pre-determined melting temperature is selected based on at least one of the type of the fresh fruit ingredient 34 and the type of the crust ingredient 26. For example, an embodiment of the kit 20 for making an apple pie having the fresh fruit ingredient 34 comprising apples and having two of the crust ingredient 26, may call for a pop-up thermometer 36 that “pops up” at a certain temperature and another embodiment of the kit 20 assembled for making a cherry pie having the fresh fruit ingredient 34 comprising cherries and having two of the crust ingredient 26 may call for a pop-up thermometer 36 that “pops-up” at a different temperature.

In the embodiment of the kit 20 for making an apple pie, the thermometer 36 is a pop-up thermometer comprising a release element 48 having a pre-determined melting temperature of greater than 190° F. Preferably, the release element 48 has a pre-determined melting temperature of from about 203° F. to about 217° F. In this embodiment, the preferred pre-determined melting temperature was determined experimentally and provides for mistake-proofed preparation of the dessert, i.e., the apple pie. Because the total amount of time the dessert is in the oven depends on when the thermometer 36 “pops-up”, the consumer does not have to know the ins and outs of their own oven to avoid undercooking or overcooking the apple pie.

In a preferred embodiment, the dessert is an apple pie and the thermometer 36 is a pop-up thermometer having a length L of about 1.0 inch. After the ingredients are assembled and the apple pie is prepared, but prior to baking the apple pie, the thermometer 36 is inserted into an apple slice. Preferably, the apple slice in which the thermometer 36 is inserted is from about 0.5 to about 3.0 inches away from the edge of the pan 24. More preferably, the apple slice in which the thermometer 36 is inserted should be of from about 1.0 to about 1.5 inches away from the edge of the pan 24.

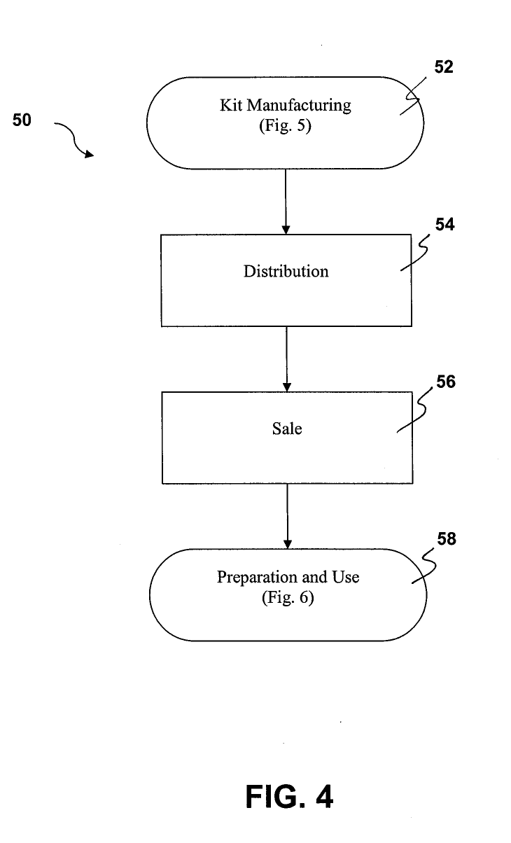

FIG. 4 is a flow chart generally illustrating the Steps of providing a kit 20 for making a non-frozen dessert. Referring to FIG. 4, a method for providing the kit 20 in accordance with one embodiment of the present invention is shown at 50. Components of the kit 20 are obtained and the kit 20 is manufactured and assembled at Step 52. The kit 20 is distributed to retailers at Step 54 and sold to a consumer at Step 56. In turn, the consumer prepares the dessert at Step 58.

Generally speaking, FIG. 4, Step 52 includes a method of manufacturing the kit 20 for making a non-frozen dessert. The method of manufacturing the kit 20 is provided. The method of manufacturing comprises the Steps of providing the pan 24, providing the crust ingredient 26, providing the filling, providing the thermometer 36 capable of measuring a temperature of the filling, and assembling the pan 24, the crust ingredient 26, the filling (and the ingredients thereof), and the thermometer 36 in the carton 22 to form the kit 20.

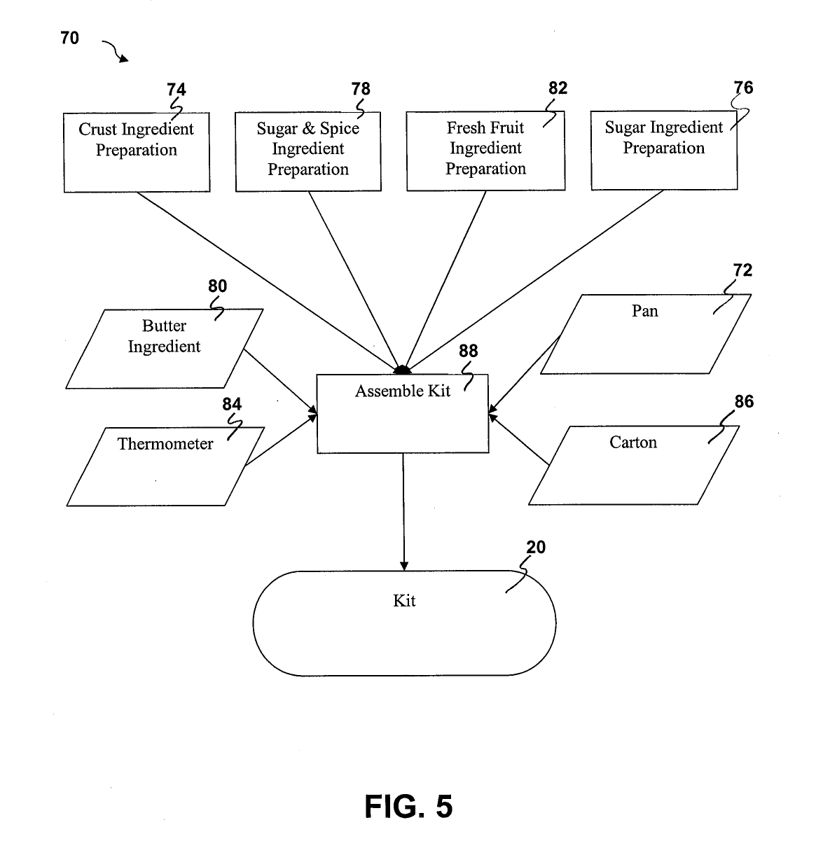

Referring now to FIG. 5, a schematic diagram generally illustrating one embodiment of the method of manufacturing the kit 20 for making a non-frozen dessert is described at 70. FIG. 5 illustrates how the kit 20 can be assembled and manufactured, as well as Steps that may have to be performed at stations in order to develop the components or ingredients, and prepare them to be packaged for distribution. In this embodiment, the method of manufacturing comprises: providing the pan 24 at Step 72; providing a crust ingredient 26 at Step 74; providing a sugar ingredient 28 at Step 76; providing a sugar and spice ingredient 30 at Step 78; providing the butter ingredient 32 at Step 80; providing the fresh fruit ingredient 34 at Step 82; providing a thermometer 36 capable of measuring a temperature of the filling at Step 84; providing the carton 22 at step 86; and assembling the pan 24, the crust ingredient 26, the filling, and the thermometer 36 in a carton 22 to form the kit 20 at Step 88. The kit 20 is assembled and packaged to minimize the time and effort that it takes to make the dessert.

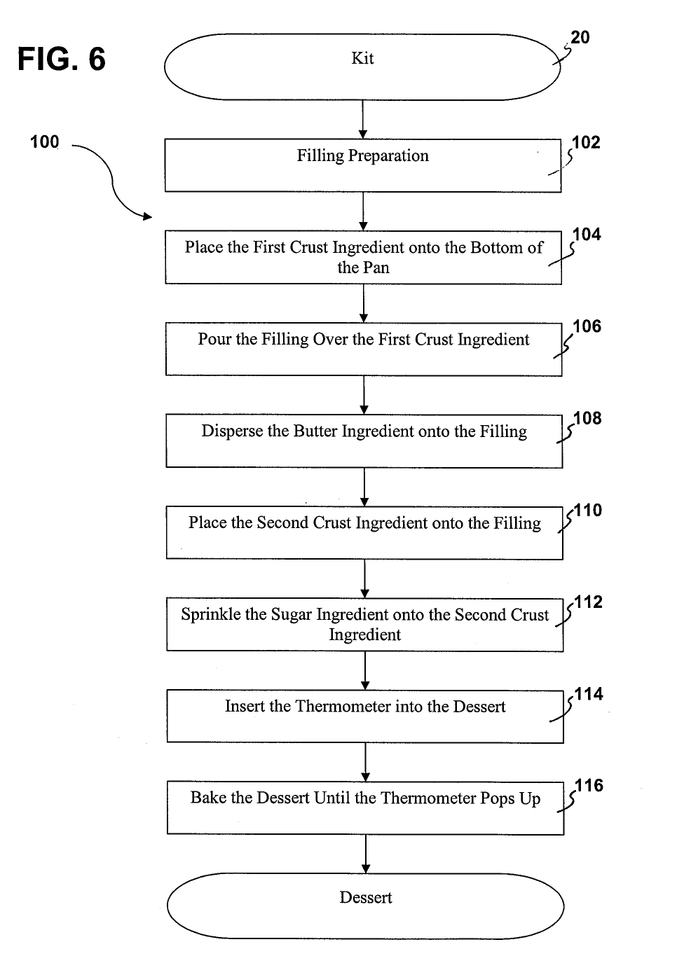

Generally speaking, FIG. 4, Step 58 includes a method of making the non-frozen dessert. Referring now to FIG. 6, one embodiment of a method of preparing and making the dessert with the kit 20 is generally shown at 100. Upon purchase of the kit 20, a consumer must prepare the dessert. Preparation of the dessert includes a number of Steps. Filling preparation at Step 102 includes: opening the package of the fresh fruit ingredient 34, e.g. apples, and pouring the fresh fruit ingredient 34 into a mixing bowl; opening the package of the sugar and spice ingredient 30 and sprinkling sugar and spice ingredient 30 across the fresh fruit ingredient 34; and mixing the fresh fruit ingredient 34 and the sugar and spice ingredient 30 thoroughly such that the sugar and spice ingredient 30 is dispersed homogeneously throughout the fresh fruit ingredient 34. The crust ingredient 26 is opened, unrolled and placed on a bottom of the pan 24 at Step 104. The mixing bowl containing a mixture of the fresh fruit ingredient 34 and the sugar and spice ingredient 30 is poured over the crust ingredient 26 at Step 106. The butter ingredient 32 is disbursed over the mixture of the fresh fruit ingredient 34 and the sugar and spice ingredient 30 at Step 108. The second package of the crust ingredient 26 is opened and the crust ingredient 26 is unrolled and placed over the mixture of the fresh fruit ingredient 34 and the sugar and spice ingredient 30 with the butter ingredient 32 thereon at Step 110. The sugar ingredient 28 is sprinkled evenly over the crust ingredient 26 at Step 112. The thermometer 36 is inserted through the crust ingredient 26 and into the filling, i.e., through the crust ingredient 26 and into a slice of apple at Step 114. The thermometer 36 is inserted into a location about 1 inch in from the side of the pan 24 to optimize results.

Once the dessert, e.g. the apple pie, is assembled, it is baked at Step 116. The dessert is baked for a period of time until the thermometer 36 “pops up” to indicate that the dessert has finished baking. As such, the dessert having fresh, homemade taste is effectively prepared in a minimal amount of time and with a minimal amount of effort.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A kit for making a non-frozen dessert capable of being transported and stored without sacrificing freshness, said kit comprising:
a carton,
a pan disposed in said carton for forming said non-frozen dessert,
a plurality of ingredients disposed in said carton for making said non-frozen dessert, said plurality of ingredients comprising:
A. at least one crust ingredient for disposing on said pan prior to baking said non-frozen dessert;
B. a filling for disposing on said crust ingredient prior to baking said non-frozen dessert, said filling comprising:
i. a sugar and spice ingredient;
ii. a butter ingredient; and
iii. a fresh fruit ingredient;
wherein said crust ingredient, said sugar and spice ingredient, and said butter ingredient are each individually packed in hermetically sealed packages; and
wherein said fresh fruit ingredient is individually packed in a vacuum sealed package and includes at least one preservative for ensuring freshness; and
a thermometer capable of measuring a temperature of said filling.

2. A kit as set forth in claim 1 further comprising a crunch crust ingredient that is individually packed in a hermetically sealed package.

3. A kit set forth in claim 1 wherein said fresh fruit ingredient is maintained at a temperature of greater than 30° F.

4. A kit as set forth in claim 3 wherein said fresh fruit ingredient comprises at least one of apple, banana, blackberry, blueberry, cherry, chocolate, lime, peach, pumpkin, strawberry, raspberry, and rhubarb.

5. A kit as set forth in claim 1 wherein said preservative comprises a carboxylic acid selected top maintain freshness of the fruit ingredient.

6. A kit as set forth in claim 5 wherein said carboxylic acid comprises a sugar acid.

7. A kit as set forth in claim 6 wherein said sugar acid is ascorbic acid.

8. A kit as set forth in claim 1 wherein said thermometer is further defined as a pop-up thermometer comprising:
a barrel defining a tubular configuration and having a closed end and an open end;
an indicator slidably disposed within said barrel;
a release element having a pre-determined melting temperature disposed within said barrel; and
a spring element disposed within said barrel movable between a compressed position and a released position, and
wherein said position of said spring element is dependent on a temperature of said release element;
wherein said release element operatively connects said spring element and said indicator;
wherein said release element holds said spring element in said compressed position at the temperature less than said pre-determined melting temperature to indicate that said dessert is in an uncooked state; and
wherein said release element releases said spring element at the temperature greater than said pre-determined melting temperature thereby extending said indicator out of said open end of said barrel to indicate that said dessert is in a cooked state.

9. A kit as set forth in claim 8 wherein said release element has a pre-determined melting temperature of greater than 190° F.

10. A kit as set forth in claim 9 wherein said release element has a melting temperature of from about 203° F. to about 217° F.

11. A kit as set forth in claim 1 wherein said thermometer has a length of from about 0.5 to about 2.5 inches.

12. A method of assembling a kit for making a non-frozen dessert capable of being transported and stored without sacrificing freshness, said method comprising the steps of:
providing a pan;
providing at least one crust ingredient that is individually packed in a hermetically sealed package;
providing a filling comprising a sugar and spice ingredient that is individually packed in a hermetically sealed package, a butter ingredient that is individually packed in a hermetically sealed package, and a fresh fruit ingredient that includes at least one preservative for ensuring freshness and is individually packed in a vacuum sealed package;
providing a thermometer capable of measuring a temperature of the filling; and
assembling the pan, the crust ingredient, the filling, and the thermometer in a carton to form the kit.

13. A method as set forth in claim 12 wherein the fresh fruit ingredient is transported and stored at a temperature of from about 30° F. to about 75° F.

14. A method as set forth in claim 12 further comprising the step of providing a crunch crust ingredient that is individually packed in a hermetically sealed package.

15. A method as set forth in claim 14 further comprising the step of assembling the crunch crust ingredient in the carton.

16. A method as set forth in claim 12 wherein the fresh fruit ingredient comprises at least one of apple, banana, blackberry, blueberry, cherry, chocolate, lime, peach, pumpkin, strawberry, raspberry, and rhubarb.

17. A method as set forth in claim 12 wherein the preservative comprises a carboxylic acid selected to maintain freshness of the fresh fruit ingredient.

18. A method as set forth in claim 12 wherein the thermometer is further defined as a pop-up thermometer comprising:
a barrel defining a tubular configuration and having a closed end and an open end;
an indicator slidably disposed within the barrel;
a release element having a pre-determined melting temperature disposed within the barrel; and
a spring element disposed within the barrel movable between a compressed position and a released position, and
wherein the position of the spring element is dependent on a temperature of the release element;
wherein the release element operatively connects the spring element and the indicator;
wherein the release element holds the spring element in the compressed position at the temperature less than the pre-determined melting temperature to indicate that the dessert is in an uncooked state; and
wherein the release element releases the spring element at the temperature greater than the pre-determined melting temperature thereby extending the indicator out of the open end of the barrel to indicate that the dessert is in a cooked state.

19. A method as set forth in claim 18 wherein the release element has a melting temperature of greater than 190° F.

20. A method as set forth in claim 18 wherein the release element has a melting temperature of from about 203° F. to about 217° F.

21. A method as set forth in claim 18 further comprising the step of selecting the release element having the pre-determined melting temperature based on at least one of a type of the fresh fruit ingredient and a type of the crust ingredient.

22. A method as set forth in claim 12 further comprising the step of selecting a pre-determined length of the thermometer based on at least one of a type of the fresh fruit ingredient and a type of the crust ingredient.
